(19) United States
(12) Patent Application Publication
(10) Pub. No.: US 2007/0154581 A1
(43) Pub. Date: Jul. 5, 2007

(54) Composition and method for enhancing or stimulating the immune system

(76) Inventors: Kalyani M. Kumar, Richmond, VA (US); Jullian Irving Grante, Fredericksburg, VA (US); Robert McClintock, Rice, WA (US)

Correspondence Address: BIRCH STEWART KOLASCH & BIRCH, PO BOX 747, FALLS CHURCH, VA, 22040-0747, US

(21) Appl. No.: 11/642,681

(22) Filed: Dec. 21, 2006

Related U.S. Application Data
(60) Provisional application No. 60/743,085, filed on Dec. 30, 2005.

(51) Int. Cl. A61K 36/185; A61K 31/716; A61K 31/315; A61K 31/095

(52) U.S. Cl. 424/777; 514/54; 514/494; 514/706

(57) ABSTRACT

The present invention relates to a composition and method for stimulating or enhancing the immune system in a human being. The composition comprises a mixture of Manapol, beta-1,3-D-glucan, arabinogalactin, elderberry extract standardized to about 30% anthocyanins, zinc gluconate and allicin. This composition, taken as a food supplement, is particularly useful against infectious diseases and acts as an anti-bacterial, anti-viral or anti-fungal agent.

The present invention is directed to a composition and method for stimulating or enhancing the immune system in the human body.

Emphasis in the medical field in recent years has grown toward the prevention of disease, rather than the treatment of disease. As an alternative to conventional pharmaceuticals, interest has grown in the use of certain food supplements for enhancing or stimulating the body's ability to ward off disease before it occurs.

The treatment protocols involved in preventing disease are based upon the belief that common etiologic factors for most, if not all disease, are due to a decrease in cellular immunity and some form of DNA alteration. The treatment goal is to work first toward cellular repair, which will improve overall cellular health through the use of nutraceuticals and the process of immune modulation. The present inventors have developed certain wellness platforms that address issues relative to women's health, men's health, children's health and senior's health, including infectious diseases.

The objective of the present inventors is prevention and wellness through intercellular and molecular medicine, instead of conventional medicine, as the key to reducing the ever-increasing cost of health care.

All cells work through an intercellular communications network. This provides the body with undetectable enhanced immunity systems to prevent disease and repair damaged cells. The inventors believe that the use of proper immune support systems aids the body's ability to self-repair.

Wellness can best be achieved with behavioral and lifestyle modifications utilizing multimodal approaches. Through the use of state of the art technology and botanical alternatives, as appropriate, this approach strives to improve the physical and emotional wellness of patients. The present inventors have identified certain botanical products, including a variety of non-pharmacological alternatives, that include nutritional and natural mineral supplementations as safe and effective alternatives.

The inclusion of prevention and wellness management by physicians as a standard of patient care will decrease the amount of prescription drugs, hospitalizations and defensive medicine-related tests, treatments and surgeries. A reduction in prescription medications will result in a substantial decrease in medical errors and will contribute significantly to lowering the cost of health care.

Toward these objectives, the inventors have found that a composition comprising the following ingredients can be employed to prevent certain diseases by stimulating or enhancing the immune system of a body. The composition of the present invention comprises a combination of Manapol, beta-1,3-D-glucan, arabinogalactin, elderberry extract (standardized to 30% anthocyanins), zinc gluconate and allicin.

### Manapol:

A component known as Manapol (a registered trademark of Carrington Laboratories, Inc., Irving, Tex.) contains acetylated mannans primarily in the form of mannose. Mannose as well as other complex carbohydrate polymers have been shown to possess immune modulating activity by binding to macrophage cells via protein receptors. Once the mannans are attached to the macrophage receptors, the body's immune system responds as it would if it were being activated naturally by foreign substances. This results in macrophage production of tumor necrosis factor-alpha, interleukin-1, and interleukin-6 as well as other cytokines and oxidants. Manapol also has demonstrated an ability to elevate the production of other immune cell populations as well as their relative activity levels.

The chemical characterization of Manapol indicates the presence of a family of very large molecular weight complex carbohydrates called acetylated mannans. These mannans have an average molecular weight of about 1 million Daltons as opposed to traditionally processed aloe comprised mostly of simple sugars with a molecular weight of around 10,000 Daltons. The total carbohydrate profile of the polymer in Manapol, as broken down and determined by GC-MS, is shown in the following table:

### Carbohydrate Profile of Manapol Powder

| Glyconutrient | Average Composition of Total Carbohydrates |
|---|---|
| Arabinose | ~2.0% |
| Rhamnose | ~2.0% |
| Fucose | ~0.5% |
| Xylose | ~4.0% |
| Mannose | ~60.0% |
| Galactose | ~12.0% |
| Glucose | ~9.0% |
| Galacturonic Acid | ~10.0% |
| Glucuronic Acid | ~1.0% |

### Beta-1,3-D-Glucan:

Beta-1,3-D-glucan is a natural complex carbohydrate that comes from the cell wall of Baker's yeast, Saccharomyces cerevisiae. It is believed that beta-1,3-D-glucan works to enhance the macrophages of the immune system so dramatically, that they seek out and destroy developing bacteria when it is opening, and thus, the bacteria do not have a chance to produce their toxins before they are engulfed by the macrophages. Highly purified beta-1,3-D-glucan can be taken orally as an insoluble particulate. It is taken up by the M-cells located within the Peyer's Patches of the intestine. Once inside the gut-associated lymphoid tissue (GALT), it is immediately engulfed by resident phagocytes, who then digest and secrete a soluble form of the molecule as they migrate throughout the immune system and other organs and tissues. The systemic release of these molecules allows them to bind to the CR3 receptors (MAC-I, CDIIb/CDI8) of certain immune cells (monocytes, macrophages, neutrophils, NK cells and others). The binding of the CR3 receptor will then set off a cascade of events relative to the immune system's mode. Observations from CR3 activation:

1. Potential hematopoiesis

2. Increased chemotaxis

3. Increased phagocytosis

4. More rapid cellular killing

5. Balance of Th2-ThI

The double binding of the CR3 by beta-1,3-D-glucan and complement of an opsonized target provide for more efficient immune cell response. Particularly notable in cancer, neutrophils are not typically able to “see” cancer cells. By employing beta-1,3-D-glucan, they can become involved in the targeting and destruction of tumor cells.

### Arabinogalactin:

Arabinogalactan is a naturally occurring, water-soluble polysaccharide found in high concentrations in the Larix genus of trees. Larch arabinogalactan (AG) modulates the beneficial cell populations associated with immune function. Arabinogalactan has been demonstrated in vivo to increase or modulate levels of immune system white blood cells, specifically monocytes/macrophages, NK cell activity and properidin (part of the complement immune system).

Larch AG has been shown to have a positive impact on the human immune system. In vitro cell studies have demonstrated that AG is one of the active immune compounds in Echinacea and that Larix AG, being a concentrated form of AG, modulates the human immune system better than Echinacea.

This immune modulation has been demonstrated in vivo by human feeding studies at the University of Minnesota, Southwest Research Institute and the University of Florida, where consumption of Larch AG led to increased levels of immune system white blood cells, specifically monocytes—the first line of defense that the human body has against human invasion from bacteria, fungi, viruses and other potentially harmful environmental factors.

The immune stimulating properties are shown to enhance natural killer class and macrophage activity in directing cytotoxicity toward cancer cells. AG also causes more interleukin secretion by macrophages.

### Elderberry Extract:

The extract from elderberries seems to be designed as a specific weapon against viruses. Particularly, the influenza virus forms tiny spikes, called hemagglutinins, which are laced with an enzyme called neuraminidase. The enzyme helps the virus to penetrate the cell walls of a healthy organism. The virus then remains in the cell, reproducing more viruses. The active ingredients in elderberries disarm the neuraminidase enzyme within 24-48 hours, preventing the viral harvest of host cell RNA and effectively halting the spread of the virus. An elderberry extract standardized to 30% anthocyanins has been found to be effective in the present invention.

### Zinc Gluconate:

Adequate zinc intake is critical for health. Zinc deficiency can affect cells of the immune system. If sufficiently significant, this may cause a reduction in the number of B lymphocytes and T lymphocytes (CD4 lymphocytes in particular) through increased apoptosis and also reduces specific functional capacity. Macrophage functions are also compromised, limiting their ability to engulf bacteria. The production and potency of several cytokines, the central messengers of the immune system, may also be affected negatively by zinc deficiency.

Zinc plays a part in the maintenance of epithelial and tissue integrity by promoting cell growth and suppressing apoptosis and through its under-appreciated role as an antioxidant, protecting against free radical damage during inflammatory responses. The RDA is only 10 mg elemental zinc, but many people in both developing and industrialized countries do not have this amount in their diet. Zinc deficiency is biochemically defined as a serum concentration of less than 9 ttmol/l. However, serum zinc concentrations may not fully reflect the physiological zinc status in an individual, and individuals with apparently normal serum concentrations may benefit from daily zinc supplements.

### Allicin:

Inhibition of certain thiol-containing enzymes in the microorganisms by the rapid reaction of thiosulfinates with thiol groups was assumed to be the main mechanism involved in the antibiotic effect. Recently, the present inventors have studied the mechanism of action of pure allicin molecules with thiol groups in more detail. This study has confirmed the ability of allicin to react with a model thiol compound (L-cysteine) to form the S-thiolation product S-allylmercaptocysteine. The identification of the thiolation product was proven by nuclear magnetic resonance as well as by mass spectroscopy.

The main antimicrobial effect of allicin is due to its interaction with important thiol-containing enzymes. In the amoeba parasite, allicin was found to strongly inhibit the cysteine proteinases and alcohol dehydrogenases, as well as the thioredoxin reductases which are critical for maintaining the correct redox state within the parasite. Inhibition of these enzymes was observed at rather low concentrations (>10 μg/mL). Allicin also irreversibly inhibited the well known thiol-protease papain, the NADP-dependent alcohol dehydrogenase from Thermoanaerobium brockii and the NAD-dependent alcohol dehydrogenase from horse liver. Interestingly, all three enzymes could be reactivated with thiol-containing compounds such as DTT, mercaptoethanol and glutathione. At concentrations that are at least a log higher (>100 μg/mL), allicin was also found to be toxic to tissue-cultured mammalian cells. As mentioned above, the significant difference in sensitivity between the microbial and mammalian cells may be explained by the much higher concentrations of glutathione possessed by the mammalian cells.

Allicin also specifically inhibits other bacterial enzymes such as the acetyl-CoA-forming system, consisting of acetate kinase and phosphotransacetyl-CoA synthetase. The inhibition is noncovalent and reversible. Acetate incorporation into fatty acids of isolated plastids was inhibited by allicin with a 50% inhibitory concentration (I ₅₀ value) lower than 10 mM. Furthermnore, allicin at bacteriostatic concentrations (0.2 to 0.5 mM) was found to partially inhibit, in Salmonella typhimurium, the DNA and protein synthesis, but the effect on RNA synthesis was immediate, suggesting that this could be a primary target of allicin action. E. coli RNA polymerase, in its alpha-subunit, contains a single sulfhydryl group which was shown to react with the monomercuric derivative of fluorescein, a specific reagent for thiol groups (fluorescein monomercuracetate). This suggests that RNA polymerase could also be a target for allicin.

The condensation product of allicin, ajoene, which has a similar oxygenated sulfur group, has been shown to inhibit the proliferation of Trypanosoma cruzi, possibly by inhibition of phosphatidylcholine biosynthesis. Ajoene has also been shown to inhibit phosphatidylcholine biosynthesis in the human pathogenic fungus Paracoccidioides brasiliensis. The inhibition capacities shown for ajoene clearly suggest that additional microbe-specific enzymes may also be targets for allicin.

It is reasonable to conclude, therefore, that the broad-spectrum antimicrobial effects of allicin (and ajoene) are due to the multiple inhibitory effects they may have on various thiol-dependent enzymatic systems. It is difficult at this stage to state which are the more lethal targets. It could very well be that the effect of allicin may be at different levels. Some enzymes such as the thiol proteases, which cause severe damage to the host tissues, may be inhibited at the lowest concentrations.

At low concentrations the inhibition of these enzymes may not be lethal, but sufficient to block the microbe's virulence. At slightly higher concentrations, other enzymes such as the dehydrogenases or thioredoxin reductases may be affected, and even partial inhibition of these enzymes could be lethal for the microorganism.

All of the above descriptions of the wide range of biological activities of allicin leads to the conclusion that this molecule is a prime candidate for therapeutic use. One commercial source of allicin is a product known as ALLISURE®, a registered trademark of Stone Island Holdings Limited of East Sussex, UK.

ALLISURE® powder has a shelf life of 30 months. Even after this time period independent tests have shown that at 36 months the powder is still able to kill MRSA (methicillin resistant Staphylococcus aureus). Provided the product is kept boxed, its shelf life will extend to past 36 months.

### Chemical Structure

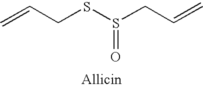

The sulphur—sulphur and sulphur—oxygen bonds are responsible for many of the beneficial properties associated with allicin. Although similar to the penicillin structure these bonds are very reactive and in fresh garlic they break down very quickly into a series of thiosulphinate components.

### Manufacturing Process

ALLISURE® powder is the result of a process which produces purified allicin liquid. It is said to be the first health food supplement to provide a 100% allicin yield, the key active ingredient of fresh garlic.

Once the garlic bulbs have been selected (approximately 5 kg per 1 million capsules), some of the batch is analyzed for alliin content using HPLC and mass spectrometry. Once this has been done, the gariic is crushed anu extra almiin frormi the same garlic is also added. As the allicin begins to form, it is physically removed from the reaction chamber by flooding the system with water. All through this phase, the temperature is carefully controlled to within 0.1° C. (this increases the yield of allicin liquid) and the whole system is kept at constant pressure. The resultant allicin liquid is analyzed by HPLC and immediately frozen for transport to the spray dryer.

At the spray drying house, the liquid is carefully added to a reaction vessel along with non-GM maltodextrin where it enters the spray dryer. The resultant powder is ALLISURE®, which is then tested microbiologically against an MRSA bacteria and HPLC. The powder is then filled into capsules.

ALLISURE® is made from fresh, raw garlic heads that are specifically selected to ensure that they contain a significant enzyme activity (allinase enzyme). Garlic heads are split into cloves, which are left unpeeled and then subjected to filtration, controlled temperature and pressure extraction and a flood reaction process designed to produce stabilized liquid allicin dissolved in water. No chemical solvents are used. The alliin amino acid in fresh garlic is subjected to complete conversion by the allinase enzyme and, to ensure that a large volume of active agent is harvested, allicin is quickly removed from the reaction system as it competes with the enzyme allinase. The volume of active agent produced is directly related to the enzymatic concentration and activity.

### Clinical Particulars

Allicin has demonstrated significant antibacterial, antifungal, larvicidal and antiviral properties. It has also shown an ability to reduce cholesterol and blood pressure as well as increasing CD4-T cell count significantly.

### Antibacterial, Antifungal, Antiviral and Larvicidal Properties

Allicin, one of the active principles of freshly crushed garlic homogenates, has a variety of antimicrobial activities. Allicin in its pure form was found to exhibit i) antibacterial activity against a wide range of Gram-negative and Gram-positive bacteria, including multidrug-resistant enterotoxicogenic strains of Escherichia coli, ii) antifungal activity, particularly against Caudida albicans, iii) antiparasitic activity, including some major human intestinal protozoan parasites such as Entamoeba histolytica and Giardia lamblia, and iv) antiviral activity. The main antimicrobial effect of allicin is due to its chemical reaction with thiol groups of various enzymes, e.g. alcohol dehydrogenase, thioredoxin reductase, and RNA polymerase, which can affect essential metabolism of cysteine proteinase activity involved in the virulence of E. histolytica.
1. Introduction

Garlic is one of the edible plants that has generated much interest throughout human history as a medicinal panacea. Wide ranges of microorganisms including bacteria, fungi, protozoa and viruses have been shown to be sensitive to crushed garlic preparations. Moreover, garlic has been reported to reduce blood lipids and to have anticancer effects. Chemical analyses of garlic cloves have revealed an unusual concentration of sulfur-containing compounds (1-3%) [1, 2].

Analysis of steam distillations of crushed garlic cloves performed over a century ago showed a variety of allyl sulfides. However, it was not until 1944 that Cavallito and his colleagues [3] isolated and identified the component responsible for the remarkable antibacterial activity of crushed garlic cloves. The compound turned out to be an oxygenated sulfur compound, which they termed allicin from the Latin name of the garlic plant, Allium sativum. Pure allicin is a volatile molecule that is poorly miscible in aqueous solutions and which has the typical odor of freshly crushed garlic [4]. Final proof of the chemical structure of allicin came in 1947, when it was shown that allicin could be synthesized by mild oxidation of diallyl disulfide [2]. The debate on the presence of allicin in crushed cloves versus its absence in odorless intact cloves was resolved after Stoll and Seebeck [5] isolated, identified, and synthesized an oxygenated sulfur amino acid that is present in large quantities in garlic cloves and which they named alliin. Alliin was found to be the stable precursor that is converted to allicin by the action of an enzyme termed allinase, which is also present in the cloves [6]. Only one isomer of alliin ((+)-S-allyl-L-cysteine-sulfoxide) was found to be present, which in itself had no antimicrobial activity. Numerous investigators studied the amounts of alliin and allicin present in different strains of garlic. Considerable variations have been reported, ranging from 2.8 to 7.7 mg/gram found in Romanian red [2].

The transformation of alliun into the biologically active allicin molecule upon crushing of a garlic clove is extremely rapid, being complete in seconds. The enzyme responsible for the lysis is alliinase, or alliin-lyase (E.C.4.4.14), a pyridoxal 3-phosphate-dependent glycoprotein consisting of two subunits 17, 81. Alliinase is present in unusually high amounts in garlic cloves, at least 10% of the total protein content (10 mg/g-fresh weight).

The gene coding for the enzyme has been cloned, and upon translation, found to consist of 448 amino acids with a protein molecular mass of 51.45 kDa and together with a carbohydrate content of 5.5-6%, gives 55000 kDa [7, 8]. Alliinase has 10 cysteine residues, all of them in S-S bridges, and their reduction, or the removal of the pyridoxal coenzyme factor, renders the enzyme inactive. Expression of a recombinant alliinase has been achieved in the baculovirus system, and although protein yields were impressive, the enzymatic activity was very poor due to difficulties with folding of the protein (Mirelman et al., unpublished results). Moreover, in the clove, allimnase is found closely associated with a lectin [9]. The site of linkage of the carbohydrate moieties of alliinase has been identified at Asp 146 [9]. Significant homology has been reported between the garlic and onion alliinases, although alliin was not detected in the latter species.

Garlic cloves are odor-free until crushed. Cross-section studies have indicated that the substrate alliin and the enzyme alliinase are located in different compartments [2, 6]. This unique organization suggests that it is designed as a potential defense mechanism against microbial pathogens of the soil. Invasion of the cloves by fungi and other soil pathogens begins by destroying the membrane, which encloses the compartments that contain the enzyme and the substrate. This causes the interaction between alliin and alllinase that rapidly produces allicin and which in turn inactivates the invader. The reactive allicin molecules produced have a very short half-life, as they react with many of the surrounding proteins, including the alliinase enzyme, and making it into a quasi-suicidal enzyme. This very efficient organization ensures that the clove defense mechanism is only activated in a very small location and for a short period of time, whereas the rest of the alliin and allinase remain preserved in their respective compartments and are available for interaction in case of subsequent microbial attacks. Moreover, since massive generation of allicin could also be toxic for the plant tissues and enzymes, its very limited production and short-lived reactivity, which is confined to the area where the microbial attack takes place, minimizes any potential self-damage to the plant.
2. Antibacterial Activity of Allicin

The antibacterial properties of crushed garlic have been known for a long time (see Table 1). Various garlic preparations have been shown to exhibit a wide spectrum of antibacterial activity against Gram-negative and Gram-positive bacteria including species of Escherichia, Salmonella, Staphylococcus, Streptococcus, Klebsiella, Proteus, Bacillus and Clostridium. Even acid-fast bacteria such as Mycobacterium tuberculosis are sensitive to garlic [10]. Garlic extracts are also effective against Helicobacter pylon, the cause of gastric ulcers [11]. Garlic extracts can also prevent the formation of Staphylococcus enterotoxins A, B, and C1 and also thermonuclease [12]. On the other hand, it seems that garlic is not effective against toxin formation of Clostridium botulinum [13].

Cavallito and Bailey [4] were the first to demonstrate that the antibacterial action of garlic is mainly due to allicin [3]. The sensitivity of various bacterial and clinical isolates to pure preparations of allicin [14] is very significant. As shown in Table I, (Mirelman et al., unpublished results) the antibacterial effect of allicin is of a broad spectrum. In most cases the 50% lethal dose concentrations were somewhat higher than those required for some of the newer antibiotics.

Interestingly, various bacterial strains resistant to antibiotics such as methicillin resistant Staphylococcus aureus as well as other multidrug-resistant enterotoxicogenic strains of Escherichia coli, Enterococcus, Shigella dysenteriae, S. flexneni and S. sonnei cells were all found to be sensitive to allicin. Allicin also had an in vivo antibacterial activity against S. fiexneri Y when tested in the rabbit model of experimental shigellosis [15].

On the other hand, other bacterial strains such as the mucoid strains of Pseudomonas aeruginosa, Streptococcus β hemnolyticus and Enterococcus faecium were iound to be resistant to the action of allicin. The reasons for this resistance are unclear. It is assumed that hydrophilic capsular or mucoid layers prevent the penetration of the allicin into the bacteria, but this needs additional study.

TABLE I
Sensitivity of various bacterial species to allicin

| | Allicin Concentration | |
|---|---|---|
| Bacterial Strain | (LD₅₀ ug/ml) | Comments |
| Escherichia coli | 15 | Sensitive to antibiotics |
| Escherichia coli | 15 | Multidrug resistant |
| | | MDR |
| Staphylococcus aureus | 12 | Sensitive |
| Staphylococcus aureus | 12 | Methicillin resistant |
| Streptococcus progenies | 3 | Sensitive |
| Streptococcus β | >100 | Clinical MDR strain |
| hemolyticus | | |
| Prloteus marbles | 15 | Sensitive |
| Proteus mirabilis | >30 | Clinical MDR strain |
| Pseudomonas aeruginosa | 15 | Sensitive to cefprozil |
| Pseudomonas aeruginosa | >100 | MDR mucoid strain |
| Acinetobacter baumanii | 15 | Clinical isolate |
| Klebsiella pneumoniae | 8 | Clinical isolate |
| Enterococcus faecium | >100 | Clinical MDR strain |

3. Antifungal Activity of Allicin

Garlic extracts also have a strong antifungal effect and inhibit the formation of mycotoxins like the aflatoxin of Aspergillus parasiticus [7]. Allicin was assumed to be the main component responsible for the inhibition of fungal growth. A concentrated garlic extract containing 34% allicin, 44% total thiosulfinates and 20% vinyldithiins possessed potent in vitro fungistatic and fungicidal activity against three different isolates of Cryptococcus neoformans. The minimum inhibitory concentration of the concentrated garlic extract against 1×10⁵ organisms of C neofornians ranged from 6 to 12 μg/mL. In addition, in vitro synergistic fungistatic activity with amphotericin B was demonstrated against all isolates of C. neoformans [18]. Pure allicin was found to have a high anticandidal activity with a minimum inhibitory concentration of 7 μg/mL [19]. Yamada and Azuma [20] report that pure allicin was effective in vitro against species of Candida, Cyptococcus, Trichophyton, Epidermphyton and Microsporum at low concentration (minimal inhibitory concentrations of allicin were between 1.57 and 6.25 μg/mL). Allicin inhibits both germination of spores and growth of hyphae [20]. The sensitivities of various clinically important yeasts to a pure preparation of allicin were determined and found to be very significant (Table II) (Mirelman et al., unpublished results). The mode of action of allicin on the fungal cell has not yet been elucidated but it is assumed to function on thiol enzymes as in other microorganisms.
4. Antiparasitic Properties of Allicin

The antiparasitic effects of freshly crushed garlic were known by many ancient cultures. Albert Schweizer used to treat people suffering from dysentery or intestinal worms with freshly crushed garlic. One of the traditional Chinese medical treatments for intestinal diseases is an alcoholic extract of crushed garlic cloves. It has been shown that Entainoeba histolytica, the human intestinal protozoan parasite, is very sensitive to allicin, as only 30 μg/mL of allicin totally inhibits the growth of amoeba cultures [21]. More recently, it has been found that at lower concentrations (5 μg/mL), allicin inhibited by 90% the virulence of trophozoites of E. histolytica as determined by their inability to destroy monolayers of tissue-cultured mammalian cells in vitro [22].

Allicin (30 μg/mL) also very efficiently inhibited the growth of other protozoan parasites such as Giardia lamblia, Leishmania major, Leptornonas colosoma and Crithidia fasciculata (Mirelman et al, unpublished results). Some allicin toxicity towards tissue-cultured mammalian cells was observed at concentrations above 100 μM [22]. Interestingly, however, at these high allicin concentrations, no damage to the mammalian cells was seen if the incubations were done in the presence of amoebic trophozoites, suggesting that the affinity of the allicin molecules is towards the parasite targets. The reason for the higher sensitivity of microbial cells to allicin than that of mammalian cells is that most of the microbial cells do not have, or have very small amounts of, glutathione (or its equivalent thiol molecules such as trypanothione) and thus lack the ability to reactivate the essential SH-enzymes that are thiolated by allicin.

TABLE II
Effect of allicin on various fungal pathogens

| | Allicin concentration | |
|---|---|---|
| Fungal strain | MIC (μg/mL) | Comments |
| Candida albicans | 0.3 | |
| Candida albicans | 0.8 | Clinical isolates |
| Candida neoformans | 0.3 | |
| Candida parapsilosis | 0.15 | |
| Candida tropicalis | 0.3 | |
| Candida krusei | 0.3 | |
| Torulopsis glabrata | 0.3 | |
| Torulopsis glabrata | 1.9 | Clinical isolates |

5. Antiviral Activity of Allicin

Fresh garlic extracts in which allicin is known to be the main active component have bhe.n shown to have in vitro and in vivo antiviral activity Among the viruses which are sensitive to garlic extracts are the human cytomegalovirus, influenza B, herpes simplex virus type 1, herpes simplex virus type 2, parainfluenza virus type 3, vaccinia virus, vesicular stomatitis virus, and human rhinovirus type 2 [23]. The allicin condensation product, ajoene, seems to have in general more antiviral activity than allicin. Ajoene was found to block the integrin-dependent processes in a human immunodeficiency virus-infected cell system [24]. Interestingly, there are some viruses like the garlic plant virus X which are resistant to the antiviral effects of garlic extracts [25].

A double blind placebo controlled study has shown significant protection from the common cold virus. Conducted by The Garlic Centre and published in Advances in Therapy, this appears to be the first serious work to show both prevention, treatment and reduction of re-infection benefits from taking ALLISURE® once daily [16].
6. A Double Blind Placebo Controlled Survey Comparing an Allicin Containing Garlic Supplement [ALLISURE®] with a Placebo

Background

The common cold is the most widespread viral infection in the world today. It is estimated that most people will suffer 2 to 5 colds per year. Over 200 different viruses cause infection and cold symptoms, the most common of which are the Rhinoviruses which account for 30-40% of adult colds. Re-infection is also very prevalent because of this wide variety of infectious viruses.

Among the viruses that are sensitive to garlic extracts are the human cytomegalovirus, human rhinovirus type 2, herpes simplex type 1 and 2 and influenza B virus. Evidence points towards allicin and its condensation product ajoene as the main components in garlic responsible for this antiviral activity.

A “cure” for the common cold would significantly reduce the number of working days lost each year due to the classic symptoms of infection which include tiredness, headaches, a runny nose, sneezing, coughing, watery eyes and a feeling of being unable to concentrate. Prevention is always better than simply treating symptoms and this survey was designed to see if a unique garlic supplement can prevent volunteers from getting a cold.

Study Objectives
1. To measure the number of colds recorded in each group as indicated by the scoring system detailed below. One group was randomized to take one ALLISURE® capsule every day and one group randomized to take one PLACEBO capsule every day for a period of 3 months.
2. As volunteers report an infection, the period of time taken to full recovery was monitored in each group.

Methodology

Following recruitment via PR in two daily newspapers, 144 participants were selected. A diary was designed for each volunteer to record progress over a 3-month period (90 days). Volunteers were asked to record general well-being on a scale of 1 to 5 every day throughout the study period.

### Symptom Measurement Scale

5=Well, no problems
4=Quite well but the occasional sneeze; no disruption to normal routine
3=Can feel a cold coming on—some minor symptoms
2=Feeling low and beginning to exhibit symptoms
1=Full cold symptoms e.g. Headache, sneezing, runny nose, tiredness

If a cold developed, then each volunteer was asked to note the number and variety of symptoms presented, the day they began to recover and the day they felt completely better.

The 144 volunteers were split into 2 groups (sex, age and garlic consumption matched—see Table III Volunteer Demographics).

TABLE III
Volunteer Demographics

| | ACTIVE | PLACEBO |
|---|---|---|
| Number of patients | 73 | 73 |
| Males | 32 | 29 |
| Females | 41 | 44 |
| Average age | 52 | 53 |
| Previously taken a garlic | 11 | 10 |
| supplement | | |

Volunteers were then randomized, using a simple random number generator and assigned to the ACTIVE (Zero) or PLACEBO (One) group. Each volunteer was then instructed to take one capsule every day with his or her main meal. This instruction follows the manufacturer's recommendation for taking a garlic supplement. Randomization codes were kept securely and were not broken until all the diaries had been returned.

Volunteers were contacted every 2 weeks to ensure that the capsules were being taken correctly and that the diary was completed daily.

### Diary Analysis

Following return of the diaries, the number of colds experienced by volunteers was counted. A cold is defined as a score of 3 which then proceeds to a score of 2 or 1 and some symptoms are experienced.

The duration of symptoms was taken as the number of days with a recorded score of 2 or 1 leading to an average recovery time ending with a score of 4 or 5 taken across all recorded colds.

### Results

The number of colds experienced in each group is shown in Table IV and the number of infected days and average number of days to a recovery is shown in Table V.

The number of colds in the ACTIVE Group was 24 and the number of colds in the PLACEBO Group was 65. This result is highly statistically significant in favor of using ALLISURE® as a cold prevention remedy. p<0.0001

The average number of days needed to recover in the PLACEBO Group was 5.63 days (366 days of infection/number of colds) whereas in the ACTIVE Group this figure was 4.63 days (111 days of infection/number of colds). p<0.0001

The number of volunteers experiencing more than 1 full cold throughout the survey period was much higher in the PLACEBO group. A total of 16 volunteers became re-infected while taking PLACEBO as compared to only 2 volunteers taking the ACTIVE.

### Diary Comments and Withdrawals

Volunteers were also asked to record any other factors that concerned them over the course of this study. Comments about the acceptability of taking capsules, side effects, smell and anything that might warrant a discontinuation of treatment were reported in the diaries kept by the volunteers and to see if further advice was required.

TABLE V
Infected days and recovery period

| TREATMENT | | | |
|---|---|---|---|
| One capsule per day | | | RECOVERY |
| With food | COLDS | INFECTED DAYS | PERIOD |
| ACTIVE (ALLISURE ®) | 24 | 111 | 4.6 |
| PLACEBO | 65 | 366 | 5.6 |

There were a total of 4 withdrawals, 3 from the ACTIVE group and 1 from the PLACEBO group. One volunteer from the ACTIVE group was withdrawn because the volunteer continued to take another garlic supplement. One volunteer from the ACTIVE group developed gout and was advised to discontinue.

TABLE VI
Frequency of re-infection
Volunteers experiencing more than 1 cold
throughout the survey period

| | |
|---|---|
| ACTIVE | 2 (Two) |
| PLACEBO | 16 (Sixteen) |

One volunteer from the ACTIVE group developed an itchy rash below the knees, which faded away after stopping the treatment.

The PLACEBO volunteer developed severe headaches and was advised to stop taking the capsules.

A total of 5 volunteers noticed a “smell” while burping after taking capsules. Four volunteers were taking ACTIVE and 1 was taking PLACEBO. However, it is not clear if they took capsules in accordance with the instructions (i.e. with their main meal).

Several volunteers taking ACTIVE reported feeling much more alert and generally healthier even though close contacts around them were falling ill. Several volunteers taking ACTIVE took them on holiday and reported avoiding a stomach upset and not getting bitten by mosquitoes.

### Conclusions

This survey is the first one to follow a double blind placebo controlled design in the area of viral disease prevention using a garlic supplement. The results are overwhelmingly in favor of allicin as a disease prevention measure. Also, in the treatment of troublesome symptoms such as a sneezing, cough and a runny nose, volunteers taking allicin recover faster. Furthermore, the data indicate a faster reduction in symptoms and recovery to full fitness. Volunteers taking the active prevention were also less likely to become re-infected from other viral strains, indicating a general improvement in the immune system.

Another important point to note is that volunteers in the ALLISURE® group took the manufacturer's recommended daily dose of 1 capsule per day indicated in the commercially available product. Many other studies published on garlic supplements, for numerous applications over the last 10 years, have often used double or triple the actual dose available in retail outlets.

This approach may represent not only a “cure” for the common cold (or at least an effective treatment), but it clearly shows that effective prevention of infection and re-infection may be gained from taking allicin on a daily basis throughout the year. The overall potential savings gained by preventing workers from taking sick leave is enormnous. The product clearly exhibits excellent antiviral activity and potential as an effective antiviral agent.
7. Anti-Histamine Activity

A pilot investigation into the use of ALLISURE® (allicin) for the treatment of hay fever (seasonal allergic rhinitis) was also carried out.

The survey was designed to determine whether a unique garlic supplement that contains only stabilized allicin could prevent the classic hay fever attack from occurring amongst volunteers who have suffered for some years. The extract ALLISURE® was chosen for this study, as it is the only product that claims to contain allicin as a starting material. Using a simple 5 point scoring system to grade the severity of any hay fever attacks, it was found that the overall average score was 3.95, indicating that allicin was able to control hay fever very well. Over 80% of volunteers reported a significant reduction in the number of challenges throughout the study period. Only 2 volunteers needed to resort to drug treatment for an attack.
The overall average score was 3.95, indicating that allicin is able to control hay fever very well.
Over 80% of volunteers reported a significant reduction in the number of challenges throughout the study period.
Only 2 volunteers needed to resort to drug treatment for a hay fever attack.
Most volunteers were impressed with the treatment and claimed that their hay fever was “much better” controlled with allicin.
Volunteers reported far fewer symptoms than they expected with big reductions in “sore eyes,” “runny nose,” “itching at the back of the throat,” “sneezing” and “tiredness”.
The volunteers found allicin easy to take and did not report any side effects. There were no reports of smell while taking the product.

Generally, the volunteers reported that allicin was easy to take and actually rather effective. Although the treatment did not work for everyone and some comments indicated that the “season” was finishing, most volunteers were extremely positive and included observations that previous drug treatment had never really removed all symptoms whereas allicin did. People were more able to go about their normal daily routine without interruption from troublesome symptoms. One gentleman reported being able to play golf 3 times a week without any problems. Another young lady was able to sit out on a fresh mown lawn for the first time since her hay fever symptoms developed in her teens. Other unsolicited comments included volunteers being able to mix and socialize without worrying about running nose and streaming eyes.

This pilot investigation shows that allicin-based supplements show an ability to prevent allergic reaction to pollen and may indeed offer a safe and natural alternative to pharmaceutical preparations. The treatment should be started as early as possible and continued throughout the season. Further work should be done to ascertain the exact degree of efficacy, but for many people, this method of treatment represents a real chance to reduce the number of compromises that hay fever sufferers have to make each year.
8. Method of Administration

Allicin capsules contain allicin powder and are adapted for oral administration. It is recommended that they should be taken with food to minimize any risk of a smell developing.

However, it is perfectly acceptable to break open the capsules and consume the powder by placing it onto or into food during preparation.
9. Undesirable Effects

The incidence of side effects while taking allicin is extremely low. Very few people report an odor while taking the product. Sensitivity can occur very infrequently and a rash is the most obvious sign. Any untoward side effects stop once the product is discontinued. Since ALLISURE® is made from fresh garlic, it can be seen to have a safety record dating back thousands of years and is unlikely to cause any problems.
10. Use in Children

Generally supplements are not recommended for children under the age of 7 years. However, provided the recommended daily dose is not exceeded, allicin can be safely taken by children aged 7 and over.
11. Pharmacological/Pharmacokinetic Properties

The allicin powder that makes up ALLISURE® is slightly acidic and as such it prefers the acid environment found in the human stomach. Since ALLISURE® does not contain any alliin or alliinase enzyme, it is impossible for the stomach acid to inactivate the allicin absorption. Therefore, a genuine 100% yield is guaranteed from each dose of ALLISURE®. All other garlic supplements rely on the body being able to produce allicin and many are imperfectly protected against attack from stomach acid. Any acid contact will completely and irreversibly inactivate aiiiinase enzyme, making production of allicin impossible.

Once absorbed, allicin breaks down as predicted empirically to form a series of thiosulphinate compounds. None of these components can be easily measured or even detected in the blood at present, although radiolabeling of allicin has been performed to confirm the expected breakdown components. One extremely beneficial component formed is ajoene, and this has also demonstrated significant antiviral properties.
12. Pharmaceutical Particulars

The active agent is allicin. Each capsule contains 300 mg of allicin powder.
13. Excipients

Typical excipients include non-genetically modified maltodextrin from maize, gum acacia and silica or other excipients as known in the art.

A preferred composition employed in the present invention, known as ImmuneStat^{X}_{R}™ is as follows:

| | Strength | Formulation |
|---|---|---|
| Manapol | | 80.00 mg |
| Beta 1,3-D-Glucan | | 25.00 mg |
| Arabinogalactin | | 50.00 mg |
| Elderberry Extract | ~30% anthocyanins | 75.00 mg |
| Zinc (as gluconate) | ~14% | 53.5 mg |
| Allicin | | 37.50 mg |

Various excipients and additives customarily used in food supplements may be included in the composition. Such excipients include, for example, vegetable cellulose, purified water, rice flour, magnesium stearate and silicon dioxide.

The formulation of the invention is normally manufactured in tablet or capsule form for oral administration. As a dietary supplement, adults can take two capsules daily or as directed by a health care professional. Higher therapeutic dosages may be beneficial for some conditions when taken as recommended by a health care professional. For children 2-10 years of age, one capsule per day is a suitable dosage.

The formulation of the invention is prepared by mixing the stated ingredients in an amount of about ±10% by weight of the components shown above.

### EXAMPLE

A preferred embodiment of the composition according to the invention is prepared by adding the following ingredients to a blending machine:

| | | |
|---|---|---|
| Manapol | 80 | mg |
| beta-1,3-D-glucan | 25 | mg |
| arabinogalactin | 50 | mg |
| elderberry extract | 75 | mg (30% anthocyanins) |
| zinc gluconate | 53.5 | mg |
| allicin | 37.5 | mg |

After thorough blending in the blending machine, 10% of the total blend is removed and subjected to the emission of a proprietary bio-energizing system utilizing sophisticated plasma generated patterns of a weak alternating field which permanently remains in the exposed substance. The treated blend is then mixed back in with the balance of the formulation and pressed or otherwise formed into tablets or capsules by techniques well known in the art for oral administration.

The composition and method of the invention are particularly useful against infectious diseases where the inventive composition acts as an antibacterial, anti-viral or anti-fungal agent. The compositions of the invention are taken as food supplements by the individual.

Other formulations can be prepared using various combinations of ingredients disclosed herein, depending upon the targeted disease or condition.

The details concerning the production and use of ALLISURE® (allicin) are generally applicable to the preparation and use of the composition of the invention. Accordingly, compositions containing various blends of the disclosed ingredients may be used effectively, depending on the condition being treated. For example, novel compositions having two or more of the listed ingredients are contemplated as being within the scope of the invention.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

### REFERENCES

[1] Darbyshire B., Henry R. J., Differences infructan content and sythesis in some Allium species, New Phytol. 87 (1981) 249-256.
[2] Koch H. P., Lawson L. D., Garlic, the science and therapeutic application of Allium sativum L. and related species, in: Retford D. C. (Ed.), Williams and Wilkins, Baltimore, 1996, pp. 1-233.
[3] Cavallito C., Bailey J. H., Allicin, the antibacterial principle of Allium sativum. Isolation, physical properties and antibacterial action, J. Am. Chem. Soc. 66 (1944) 1944-1952.
[4] Block E., The chemistry of garlic and onion, Sc. An. 252 (1985) 94-99.
[5] Stall A., Seebeck E., Chemical Investigations of alliin, and the specific principle of garlic, Adv. Ensymol. 11 (1951) 377-400.
[6] Ellmore G. S., Feldberg R. S., Alliin lyase localization in bundle sheaths of garlic clove (Allium sativum), Am. J. Bat. 81 (1994) 89-94.
[7] Rabinov A., Xiao-Zhu Z., Grafl G., Galili G., Mirelman D., Alum lyase (allinase) from garlic (Allium sativum); Biochemical characterization and cDNAcloning, Appl.Biochem.Biotechnol. 48 (1994) 149-171.
[8] Van Damme 5.3.24., Smeets K., Torrekens S., Van Leaven F., Peumans W. J., Isolation and characterization of alliinase cDNA clones from garlic (Allium sativum L.) and related species, Eur. J. Biochem, 209 (1992) 751-757.
[9] Rabinkov A., Wilchek M., Mirelman D., Alumnae (alum lyase) from garlic (Allium sativum) is glycosylated at ASN146 and forms a complex with a garlic mannosespecific lectin, Glycoconj. 3. 12 (1995) 690-698.
[10] Uchida Y., Takahashi T., Sato N., The characteristics of the antibacterial activity of garlic, Jpn J. Antibiotics 28 (1975) 638-642.
[11] Celiini L, Di Campli B., Masuli M., Di Bartolomeo S., Aliocati N., Inhibition of Helicobacter pylori by garlic extract (Allium sativum), FEMS Immenol. Med. Micrbiol 13 (1996) 273-277.
[12] Gonzales-Fandos F., Garcia-Lopez Mi., Sierra Mi., Otero A., Staphylococcal growth and enterotoxins (A-D) and thermonuclease synthesis in the presence of dehydrated garlic, J. Appl. Bacteriol. 77 (1994) 549-552.
[13] Girnenez M A., Solanes R E., Gimeriez D. F., Growth of Clostridiumn botulinum in media with garlic, Rev. Argent. Microbioi. 20 (1988) 17-24.
[14] Rabinov A., Miron T., Konstrantinovski L., Wilchek M., Mirelman D., Weiner L., The mode of action of allicin: trapping of radicals and interaction with thiol containing proteins, Biochim. Biophys. Acts 1379 (1998) 233-244.
[15] Chowdhury A K., Ahsan M., Islam S N., Ahmed Z. U., Efficacy of aqueous extract of garlic and allicin in experimental shigellosis in rabbits, Ind. J. Med. Res 93 (1991) 33-36.
[16] Josling P D, Double blind placebo controlled evaluation of Allisure in preventing and treating influenza virus—The Common Cold. Alternative in Therapy (In press).
[17] Lawson L D., The composition and chemistry of garlic cloves and processed garlic, in: Koch H. P., Lawson L. D. (Eds.), Garlic: the science and therapeutic application of Allium sativum L., Williams and Wilkins, Baltimore, 1996, pp. 37-108.
[18] Davis L B., Shen 3., Rover R. E., In vitro synergism of concentrated Allium sativum extract and amphotericin B. against Cryptococcus neoformans, Planta Med. 60 (1994) 546-549.
[19] Hughes E. G., Lawson L. D., Antimicrobial effects of Allium sativum L. I garlic) Allium ameelopratrum (elephant garlic) and Allium cepa L. (Onion) garlic compounds and commercial garlic supplement products, Phytothet. Res. 5 (1991) 154-158.
[20] Yamada Y, Azuma K., Evaluation of the in vitro antifungal activity of allicin, Antimicrob. Agents Chemother. [1] (1997) 743-749.
[21] Mirelman D., Motsheit D., Vaton S., Inhibition of growth of Enantomoeba histolytica by Allicin the active principle of garlic extract (Alliuum sativum), J. Infect. Dis. 156 (1987) 243-244.
[22] Ankri S., Miron T., Rabinkov A., Wilchek M., Mirelman D., Allicin from garlic strongly inhibits cysteine proteinases and cytopathic effects of Entamoeba histolyt ica, Antimicrob. Agents Chemother. 10 (1997) 2286-2288.
[23] Tsai Y, Cole L. L., Davis L. E., Lockwood S. J., Simmons V., Wild G. C., Antiviral properties of garlic: in vitro effects on influenza B, herpes simplex and coxsackie viruses, Planta [22] Med. 5(1985) 460-461.
[24] Tatatintsev A. V., Vrzhets P. V., Ershov D. E., Turgiev A S., Karamov E. V., Kornilaeva G. V., Makarova T N., Fedorov N A., Varfolomeev S. D., The ajoene blockade of inter independent processes in an HIV-infected cell system, Vestn. Ross. Akad. Med. Nauk. 11(1992) 6-10.
[25] Song S. I., Song J. T., [23] Chang M. U., Lee 3.5. Choi Y D., Identification of one of the major viruses infecting garlic plants, garlic virus X, McI. Cells 7 (1997) 705-709.

1. A food supplement composition comprising Manipol, beta-1,3-D-glucan, arabinogalactin, elderberry extract standardized to about 30% anthocyanins, zinc gluconate and allicin.

2. The composition of claim 1, containing about 22.5 to 27.5% by weight of Manipol.

3. The composition of claim 1, containing about 7 to 8.6% by weight of beta-1,3-D-glucan.

4. The composition of claim 1, containing about 15 to 17% by weight of arabinogalactin.

5. The composition of claim 1, containing about 11.6 to 11.8% by weight of allicin.

6. The composition of claim 1, containing about 23 to 23.5% by weight of elderberry extract.

7. The composition of claim 1, containing about 16.5 to 16.8% by weight of zinc gluconate.

8. The composition of claim 1, formulated as a tablet or capsule for oral administration.

9. A method for stimulating or enhancing the immune system in a human being which comprises administering an effective amount of a composition according to claim 1 to a human being.

10. The method of claim 9, wherein said composition is formulated as a tablet or capsule for oral administration.

11. A food supplement composition comprising a mixture of at least two ingredients selected from the group consisting of Manipol, beta-1,3-D-glucan, arabinogalactin, elderberry extract standardized to about 30% anthocyanins, zinc gluconate and allicin.

12. A method for stimulating or enhancing the immune system in a human being which comprises administering an effective amount of a composition according to claim 11 to a human being.

13. The method of claim 12, wherein said composition is formulated as a tablet or capsule for oral administration.
